(19) United States
(12) Patent Application Publication
(10) Pub. No.: US 2004/0161862 A1
(43) Pub. Date: Aug. 19, 2004

(54) Method of visualization and quantification of biopolymer molecules immobilized on solid support

(76) Inventors: Valeri V. Golovlev, Oak Ridge, TN (US); Ye Sun, Knoxville, TN (US); Michael P. McCann, Louisville, TN (US); Wen-Hua Fan, Knoxville, TN (US)

Correspondence Address: LUEDEKA, NEELY & GRAHAM, P.C., P O BOX 1871, KNOXVILLE, TN, 37901, US

(21) Appl. No.: 10/776,882

(22) Filed: Feb. 11, 2004

Related U.S. Application Data
(60) Provisional application No. 60/448,175, filed on Feb. 15, 2003.

(51) Int. Cl. G01N 33/553

(52) U.S. Cl. 436/525

(57) ABSTRACT

A method and kit are provided for visualization of a latent pattern of molecular structures on a substrate surface. The method is comprised of developing the substrate in a solution of colloidal particles. A detectable change on the substrate surface is brought about as a result of the precipitation of the colloidal particles. The invention also provides compositions and kits for practicing the method of the present invention. Further, the invention provides methods of capturing images of the substrate surface for visualization and quantification of the molecular structures.

### CROSS-REFERENCE TO RELATED APPLICATION

This patent application claims benefit of priority of provisional application U.S. Ser. No. 60/448,175, filed Feb. 15, 2003.

### TECHNICAL FIELD OF THE INVENTION

The present invention relates to the field of bio-polymer analysis and detection which is of interest in biomedical research, genetic studies and disease diagnosis, toxicology tests, forensic investigation, agriculture and pharmaceutical development.

### BACKGROUND OF THE INVENTION

Nucleic acid hybridization has become an increasingly important technology for DNA analysis and gene expression studies. For example, DNA and RNA hybridization techniques are very useful for detecting, identifying, fingerprinting, and mapping molecular structures. Recently developed combinatorial DNA chips, which rely on the specific hybridization of target and probe DNA on a solid surface, attracted tremendous interest from the scientific and medical communities. Although the study of gene activity and molecular mechanisms of disease and drug effects has traditionally focused on genomics, recently proteomics has introduced a very valuable complimentary approach to study the biological functions of a cell. Proteomics involves the qualitative and quantitative measurement of gene activity by detecting and quantifying expressions at the protein level, rather than at the messenger RNA level. Multianalyte assays, also known in the art as “protein chips”, involve the use of multiple antibodies and are directed towards assaying for multiple analytes. The approach enables rapid, simultaneous processing of thousands of proteins employing automation and miniaturization strategy introduced by DNA microarrays.

An attractive feature of microarray technology for genomic applications is that it has the potential to monitor the whole genome on a single chip, so that researchers can have a complete picture of the interaction among thousands of genes simultaneously. Possible applications of DNA microarrays include genetic studies, disease diagnosis, toxicology testing, forensic investigation, and agriculture and pharmaceutical development. Growing applications for microarrays creates new demands for reducing the complexity and improving the detection sensitivity of DNA chips.

Currently, the most common approach to detect DNA bound to a microarray is to label it with a reporter molecule that identifies DNA presence. The reporter molecules emits detectable light when excited by an external light source. Light emitted by a reporter molecule has a characteristic wavelength, which is different from the wavelength of the excitation light, and therefore a detector such as a Charge-Coupled Device (CCD) or a confocal microscope can selectively detect a reporter's emission. Although the use of optical detection methods increases the throughput of the sequencing experiments, the disadvantages are serious. Incorporation of a fluorescent label into a nucleic acid sequence increases the complexity and cost of the entire process. Although the chemistry is commonplace, it necessitates additional steps and reagents for fluorescent labeling, and can be accomplished only with specialized expensive equipment for detection of weak fluorescent signals.

Autoradiography is another common technique for the detection of molecular structures. For DNA sequence analysis applications, oligonucleotide fragments are end labeled, for example, with ³²P or ³⁵S. These end labeled fragments are then exposed to X-ray film for a specified amount of time. The amount of film exposure is determined by densitometry and is directly related to the amount of radioactivity of the labeled fragments adjacent to a region of film.

The use of any radioactive label has several disadvantages. First, the use of radioactive isotopes increases the risk of workers acquiring mutation-related diseases. As such, precautions must be implemented when using radioactive markers or labels. Second, the need of an additional processing step and the use of additional chemical reagents and short-lived radioisotopes increases the cost and complexity of this detection technique.

While a large number of detection methods for use with nucleic acids and protein arrays have been described in patents and in the scientific literature, virtually all methods set forth in prior art contain one or more inherent weaknesses. Some lack the sensitivity necessary to accomplish certain tasks. Other methods lack the recognition specificity due to imposing non-optimal conditions for forming probe-target duplexes. Still others are expensive and difficult to implement or present health safety concern for workers, who implement these techniques.

Thus, there is a need for an improved method and kit for visualization of molecular structures, which said method is quantitative, sensitive, and simple to implement. There is also a need for an improved method for visualizing a latent pattern of molecular structures on solid support, which does not required chemical modification of the molecular structure to make it detectable.

### Nomenclature

Unless defined otherwise, all technical and scientific terms used above and throughout the text have the same meaning as commonly understood to one of ordinary skill in the art to which this invention belongs.

The following definitions are provided to facilitate a clear understanding of the present invention. The term “molecular structure” refers to a macro-molecule, including organic compound, antibody, antigen, virus particle, metal complex, molecular ion, cellular metabolite, enzyme inhibitor, receptor ligand, nerve agent, peptide, protein, fatty acid, steroid, hormone, narcotic agent, synthetic molecule, medication, nucleic acid single-stranded or double-stranded polymer and equivalents thereof known in the art.

The term “bound molecular structures” or “duplex” refers to a corresponding pair of molecules held together due to mutual affinity or binding capacity, typically specific or non-specific binding or interaction, including biochemical, physiological, and/or pharmaceutical interactions. Herein binding defines a type of interaction that occurs between pairs of molecules including proteins, nucleic acids, glycoproteins, carbohydrates, hormones and the like. Specific examples include antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/ligand, etc.

The term “sample substance” refers to a media, often a liquid media, which was prepared for the purpose of analysis and establishing (a) the presence or absence of a particular type of molecular structure; (b) the presence or absence of a plurality of molecular structures; (c) the presence or absence of specific groups of molecular structures; (d) the presence or absence of a specific group on a molecular structure of interest.

The term “target molecular structure” or “target” refers to a molecular structure whose presence or absence in a sample substance needs to be established.

The term “target group” refers to a portion of a molecular structure whose presence or absence in a molecular structure needs to be established.

The term “probe molecular structure” or “probe” refers to a molecular structure of known nature, which said probe is capable of binding to a particular type of target molecular structure or to any agent from a specific class of molecular structures. Said probe is used to witness the presence of the corresponding target molecular structure in a sample substance.

The terms “solid support” and “substrate” are used interchangeably and refer to a structural unit of any size, where said structural unit or substrate is having a flat surface suitable for immobilization of probe molecular structures and said substrate made of a material such as, but not limited to, glass, fused silica, and synthetic polymers.

It must be noted that as used herein and in the appended claims, the singular forms “a”, “and”, and “the” include plural referents unless the context clearly dictates otherwise. Thus, for example, reference to “a molecular structure” may include a plurality of macro-molecules, including organic compounds, antibodies, antigens, virus particles, metals, metal complexes, ions, cellular metabolites, enzyme inhibitors, receptor ligands, nerve agents, peptides, proteins, fatty acids, steroids, hormones, narcotic agents, synthetic molecules, medications, nucleic acid single-stranded or double-stranded polymers and equivalents thereof known to those skilled in the art, and so forth.

### SUMMARY OF THE INVENTION

The present invention provides an improved method and kit useful for detecting, identifying, fingerprinting, and mapping molecular structures. In accordance with the present invention, the method is capable of simultaneously detecting multiple molecular structures of different type immobilized on solid support in predetermined test sites. In accordance with the present invention colloidal particles made of specific materials and carrying a net electric charge are used to visualize and characterize the quantity of target molecular structures on the surface of solid support. The method and kit provided herein substantially eliminates or reduces the disadvantages and problems associated with devices and methods known from prior art.

The method of the present invention is particularly beneficial for the detection of biopolymer materials immobilized on the surface of solid support, including DNA, RNA, natural and synthetic polynucleotides and polypeptides, proteins and the like known in the art. The method of the present invention allows visualizing and quantitatively characterizing probe-target complexes on the substrate surface by exposing and developing the substrate in a solution of colloidal particles.

Although in order to provide better understanding of the present invention examples of using gold colloidal particles for visualization and quantification biopolymers will be presented, it is appreciated that in the method of present invention colloidal particles of other materials can be used including metals and transitional elements (for instance, Au, Ag and Si), oxides (for instance, Al₂O₃, SiO₂, Fe₂O₃ and the like) and polymers (for instance, polyethylene and its derivatives, polyethylene terephthalate (PET) and its derivatives, polyacrylamide and its derivatives, polymethacrylate and its derivatives. polysterene/divinylbenzene and its derivatives, and the like known in the art).

The method of the present invention is based on the observation that colloidal particles of some materials, and particularly gold colloidal particles, bind to the surface of a solid support when specific chemical groups are presented on said surface. Now considering gold particles as an example, it is appreciated that the colloidal particles of other materials can be used in a similar manner as disclosed herein below. In an aqueous solution, colloidal gold particles normally carry a negative electric charge and show high affinity for positively charged chemical groups, though negatively charged chemical groups on the surface may repel the particles. To maximize the affinity of colloidal particles to a specific surface or chemical groups, said colloidal particles could be coated with different modifiers, such as small organic molecules, peptides and proteins. When attached to the particle, the modifier(s) adjust particle net electric charge and, in this fashion, contributes to higher affinity of the particle to the negatively or positively charged chemical groups of the probe and target molecules on the substrate surface.

The colloidal particles precipitate on the surface and cover it with a layer, where the density of the particles in said layer represents the density of attracting and repelling chemical groups on the surface. Indeed, for colloidal particle of size of 2 nm or larger, the net force that attracts or repels the particle to or from the surface represents the average force from all chemical groups and electric charges on the surface in the area comparable to the size of the colloidal particle. The colloidal particles are attaching to the surface when attraction dominates and repels from it when the repulsion prevails. The density of colloidal particles on the surface is related to the net number of attractive and repulsive groups at a corresponding location on the surface. This particular mechanism of attaching colloidal particles to the surface is presented herein in order to provide a better understanding of the present invention and is given by way of illustration, and not be a way of limitation. It is appreciated that the method of the present invention is not bound to any particular assumption or theory of the mechanism of interaction of the surface and the colloidal particle, and said method can be practiced by many different ways. Various other embodiments and variations to the preferred embodiments will be apparent to those skilled in the art and may be made without departing from the spirit and scope of the following claims.

The method of the present invention is different from the conventional detection techniques and using conjugates, in which attaching a label to the surface is driven by a specific, one-to-one interaction of homologous fragments of the probe and target molecules. Indeed, in the method of the present invention the attracting colloidal particles are driven by a non-specific, one-to-many interaction, in which the binding force applied to a single colloidal particle represents an average of many attracting and repelling forces from many chemical groups on the surface. A new and unexpected result of the present invention vs. the methods known in the art is that non-specific attaching of colloidal particles to the surface provides a sensitive and convenient approach for visualization and quantification of probe and target molecules on the surface. Yet, another new and unexpected result of the present invention is that non-specific attachment of colloidal particles to the site of the interest on the surface can be accomplished without chemical modification of target or probe molecules. An overall advantage of the method of the present invention is seen as providing a means for overcoming the drawbacks of the methods known in the art and introducing a new method for visualizing biopolymer molecules immobilized on a surface. This new method is quantitative, more sensitive, does not required chemical modification of probe or target molecules for detection, does not interfere with the binding/hybridization process of probe and target molecules, can be implemented with a large variety of surface immobilized molecular structures, and can be carried out using inexpensive detection equipment such as an optical scanner, an optical microscope equipped with a camera, and various photoequipment for capturing still and video images.

Another aspect of the invention is a kit for the practice of the method. The kit comprises multiple containers having appropriate amounts of reagents necessary to practice the method as follows: a container containing a suitable colloidal solution; a container containing capping solution for blocking the substrate prior to development in a colloidal solution; a container or attachable chamber suitable to carry out hybridization or a binding reaction; and a container suitable for washing the substrate by dipping in or rinsing with a washing buffer. Additionally, the kit can include a set of substrates suitable for immobilization of probe molecular structures, i.e., producing microarrays, and a cassette for capturing diffuse reflectance from a transparent substrate when using an optical scanner or camera.

For quantification of the hybridized target molecules, surface of the solid support (i.e., microarray) covered by colloidal particles can be analyzed and density of the bound colloidal particles can be measured using conventional optical techniques and a suitable image-capturing apparatus. Here, the suitable image-capturing apparatus can include any device of plurality of devices capable of acquiring specular and diff-use reflection from the surface of interest, and most preferably, includes flatbed scanners. The resolution of the image-capturing device must be sufficient to identify optical response from individual test sites on the surface of the substrate. Most preferably, the image-capturing device must be able to digitize the captured image and transfer the image to a computer for storage and further analysis. It is appreciated that image of the same area of the substrate can be captured multiple times for averaging, reducing noise, color manipulations, filtering and performing other image-processing operations known to one skilled in the art. Specialized software can be implemented for obtaining quantitative characteristics of the optical response from each individual test site on the substrate. These quantitative parameters can be used to quantify the distribution of colloidal particles precipitated on the substrate and accordingly to measure the quantity of molecular structure of interest in corresponded site(s) of the substrate.

### DRAWINGS

In order to more fully understand the manner in which the above-recited advantages and other objectives of the invention are obtained, a more particular description of the invention described above will be illustrated in the appended drawings. Understanding that these drawings depict only typical embodiments of the invention and are not to be considered limiting of its scope, the invention is further explained and illustrated with additional specificity and detail through the use of the accompanying drawings in which:

FIG. 1(A): schematic of diffusely and specular reflected light from a substrate illuminated by an external light source, where 1-1 is the substrate, 1-2 is the colloidal particles precipitated on the substrate surface;

FIG. 1(B) is the schematic of detection of the specular reflected light from the substrate surface; here 1-3 is the light absorbing paint on the back of the substrate; the sensor of image capturing device is placed in direction in which specular reflected light is propagated;

FIG. 1(C) is the schematic of detection of the diffusely reflected light from the substrate surface; here the sensor of image capturing device is placed in a direction in which only diffuse reflected light is propagated;

FIG. 1(D) is schematic of detection of the diffusely reflected light from a transparent substrate surface; 1-4 here is the light absorbing screen placed behind the substrate which screen absorbing and reducing intensity of light components other than scattered reflection from the front surface of the substrate; the light absorbing screen normally placed behind the substrate on a distance exceeding the focal depth of an image capturing device used to acquire image of the substrate surface.

FIG. 2: Poly-L-lysine molecules immobilized on a substrate are visualized using the method of the present invention. The substrate is developed in solution of 250 nm gold particles. The density of the immobilized molecules decreased from top to bottom and from left to right and is (a) 1 ng/μl, (b) 0.8 ng/μl, (c) 0.6 ng/μl, (d) 0.5 ng/μl, (e) 0.4 ng/μl, (f) 0.3 ng/μl, (g) 0.2 ng/μl, (h) 0.1 ng/μl, and (i) 0.05 ng/μl.F

FIG. 3: Amplitude of the diffusely reflected light at the center of a spot as shown in FIG. 2 vs. the density of poly-L-lysine molecules immobilized in corresponding site of the substrate.

FIG. 4: M13mp8 Phage DNA was immobilized on the Mylar substrate activated with 0.1% solution of γ-aminopropyltriethoxylsilane. A latent pattern of DNA spots on the substrate surface was produced by pipetting 1 μl of solutions contained 1 ng/μl (column #1), 10 ng/μl (column #2), and 100 ng/μL (column #3) of DNA. DNA molecules are captured by positively charged amino groups on the substrate surface. (A) To visualize the latent pattern, the substrate was developed in solution of 250 nm non-modified, i.e., negatively charged gold particles for 15 min at room temperature. The particles, which are carrying a net negative charge, are attracted by positively charged amino groups and precipitate on the substrate except for the spots, where DNA molecules are located. Here, the negatively charged DNA molecules repel negatively charged colloidal particles from corresponded sites of the substrate surface; and (B) shows an example of visualization of immobilized DNA molecules using 250 nm gold particles modified to carry a net positive charge. Positively charged colloidal particles were prepared by adding 80 μl of 0.01% poly-L-lysine (Sigma-Aldrich) to 1 ml of gold colloid at concentration 3.6×10⁸ particles/ml. The mixture was incubated at room temperature at constant shaking for 2 hours. The solution was centrifuged to precipitate colloidal particles and the natant carrying the residual unbound poly-L-lysine was discarded. A pellet of colloidal particles was resuspended in distilled deionized water to the original concentration of 3.6×10⁸ particles/ml and this colloidal solution was used to develop the latent pattern of the DNA molecules on the substrate. The development was carried out by dipping the substrate into the colloidal solution for 15 min at room temperature. When developing was completed, the substrate was washed gently using distilled deionized water, dried by centrifugation and scanned using Epson Perfection 3200 flatbed scanner.

FIG. 5: Detection of protein A (spots #1) and ImG (spots #2) immobilized on amino-modified Mylar substrate, activated by treatment with 0.1% solution of γ-aminopropyltriethoxylsilane. The substrate was developed by dipping for 5 min in a solution of 250 nm negatively-charged gold particles at a concentration 1×10⁸ particles/ml. Spots #3 are negative (empty) control and in spots #4 immobilized protein A was exposed to ImG.

FIG. 6(A)—Visualization of hybridized oligonucleotides on a substrate by the method of the present invention. A set of 5 oligonucleotides each 50 bases long was printed on a surface of amino-modified glass substrate in an array of 8×8 spots with a layout shown in FIG. 6(B). Oligonucleotide sequence, concentration of the printing solution and location of corresponded spots are listed in Tables I and II hereinbelow. The microarray was hybridized with four homologous target oligos and the latent pattern of hybridized and non-hybridized molecular structures on microarry surface was visualized using a solution of 250 nm positively charged colloidal particles.

### DESCRIPTION OF THE PREFERRED EMBODIMENTS.

The following describes steps involved in the detection method of the present invention, materials, amounts of reagents and other variables such as time and temperature of the steps. The following also describes how a quantitative measurement of the number of biopolymer molecules of interest can be carried out. In order to provide a better understanding of the present invention specific examples are given by way of illustration and not by way of limitation.

1. For the purpose of immobilization of probe biopolymer molecules on the surface of a solid support, said surface of solid support can be activated using techniques of surface activation known in the art including, but not limited to, activation using amine reactive chemistries, sulfhydryl reactive chemistries, carbonyl reactive chemistries, hydroxyl reactive chemistries, active hydrogen reactive chemistries, silanation chemistries, and the like, see G. T. Hermanson, et al, “Immobilized Affinity Ligand Techniques”, Academic Press (1992), all activation techniques are included herein by reference. Equally acceptable, surface preparation for immobilization of probe biopolymer may include treating and coating the surface by mediating binding agents such as poly-L-lysine, poly-1-glutamic acid, poly-1-aspartic acid, glycine, alanine, cysteine and the like.

2. Once activated, the surface of solid support can be used for immobilization of probe biopolymer molecules by following known techniques and protocols for immobilization of nucleic acids, DNA, RNA, and proteins, which also include antibodies and antigens and the like, see Hegde, P. et al. “A Concise Guide to cDNA Micro-Array Analysis”, BioTechniques 29, 549-562 (2000); Rehman, et al, “Immobilization of Acrylamide-modified oligonucleotides by copolymerization”, Nucleic Acids Res., v. 27, p.649-655; Eisen, et al, “DNA Arrays of Gene Expression”, Methods Enzymol., v. 303, p. 179-205, all immobilization techniques and protocols are incorporated herein by reference. Immobilization of probe biopolymers results in allocation of known types of probe agents at known locations on the surface. Also true is that the specific location on the surface can be used to identify the type of probe biopolymer molecules at that specific location. An acceptable density of immobilized probe molecular structures ranges from about 0.01 ng/mm² to 50 ng/mm², preferably from about 0.05 ng/mm² to about 10 ng/mm² and more preferably from about 0.1 ng/mm² to about 1 ng/mm².

3. When immobilization of the probe molecules on the surface has been completed, an additional step of blocking the surface of the solid support can be performed. Blocking prevents non-specific binding of target molecules to the solid support. The blocking also can be used to allocate specific chemical groups on the surface for maintaining desirable positive or negative net surface charge on the substrate surface. Different reagents can be used to block or cap an activated solid support, whereby blocking agents couple and block residual active sites and essentially eliminate said sites from non-specific binding of target biopolymers. Common blocking or capping agents can include glycine, ethanolamine, tris(hydroxymethyl)aminomethane, mercaptoethanol, mercaptoethylamine, cysteine, acetic anhydryde, succinic anhydride, albumine, sodium borohidrade, ammonium chloride, sodium acrylate, etc. Maintaining desirable electric charge on the surface can be achieved by using poly-L-lysine, anionic and cathionic polymers, for instance, PDDA, amino- and mercapto-silane derivatives, etc. One skilled in the art will adjust concentration and time to optimize blocking treatment to a specific type of chemistry used to activate the solid support.

4. The binding or hybridization operation is performed during which the solid support with immobilized probe biopolymers is exposed to a solution of target molecules. Target molecules bind to the homologous probes on the surface of the solid support. Specificity of the binding operation can be enhanced by optimizing pH, ionic strength, and temperature of the buffer solution in which binding or hybridization is performed. Duration of the binding operation is another important parameter, which can be used for maximizing specificity of binding process.

5. The binding operation usually is completed when probe or analyte molecules available for binding are exhausted. However, in some embodiments of this invention the binding operation can be terminated after a predefined reaction time by replacing the hybridization solution with a solution, which is free of analyte/target molecules.

6. When binding is complete, an optional additional step of modification of the surface of solid support can be implemented by exposing the surface to reagents such as small organic molecules, polynucleotides, peptides and proteins, thus causing these reagents to be immobilized on the surface. This optional step modifies the affinity of the surface to the colloidal particles, which improves the visualization and measurements on the surface.

7. Yet, in another embodiment of the present invention after completion of binding of probe and target molecules the substrate is exposed to a solution containing one or more enzymes, which enzymes are capable to digest unbound molecular structures on the substrate surface. Examples of such enzymes includes S1 nuclease, Mung Beam Nuclease, and Exonuclease I, which provided herein by way of illustration and not by way of limitation. Now considering S1 nuclease as an example, the S1 nuclease is isolated from Aspergillus oryzae and is available from various vendors (see, for instance, Startagene, Promega, etc.). S1 degrades single-stranded nucleic acids, although double-stranded RNA, DNA and RNA-DNA hybrids are resistant to S1 nuclease digestion unless large excess of enzyme is used. To achieve satisfactory results one skilled in the art will adjust concentration of enzyme solution, temperature and time of treatment to obtain desirable removal of single-stranded probe molecules. In this embodiment of the present invention the enzymatic digestion of unbound probe molecules provides better discrimination between bound and unbound molecular structures when both probe and probe-target complexes can initiate a detectable precipitation of colloidal particle. This embodiment of the present invention is especially beneficial for identification of a presence of specific molecular structures in a sample substance, although it also can be used to identify an absence of specific molecular structures in a sample substance.

8. Alternatively, in yet another embodiment of the present invention after completion of binding of the targets and probes immobilized on the substrate the substrate surface is exposed to a solution containing one or more enzymes, which enzymes are capable to digest preferably bound probe-target molecular structures on the substrate surface. One example of such enzyme is Exonuclease III (from E. coli) which provided herein by way of illustration and not by way of limitation. Exonuclease III digest double-stranded DNA and can be used for enzymatic digestion of bound probe molecules. This enzymatic treatment allows identification of sites where no binding reaction occurs most preferably due to the absence of corresponded target molecular structures in a sample substance. Therefore, this embodiment of the present invention is most preferable for identification of absence of specific molecular structures in a sample substance, although it also can be used to identify a presence of specific molecular structures in a sample substance.

9. During the binding and post binding treatment disclosed herein above a latent pattern of molecular structures is formed on the substrate surface. This pattern now can be visualized by exposing, i.e., developing the substrate in a solution of colloidal particles. During the development step particles are bound to the substrate thereby producing a thin layer of colloidal material on the surface. The density of colloidal material varies from site to site following the pattern of molecular structures on the surface. Therefore, by measuring the density of colloidal material on the surface it is possible to identify the location and also it is possible to measure the quantity of probe-target complexes on corresponding sites of the surface. The concentration of colloidal particles in solution and the temperature influences the rate of development of the image. While solutions that are used may be at a starting temperature of about 0° C. or even below, the development temperature is generally maintained in the range of about 1° C. to about 90° C. The results from 4° C. to 50° C. depending on the nature of the sample, appears preferable. Temperatures below 20° C. can also be used to prevent denaturation of probe-target complexes providing latent pattern development is controlled. The temperature, if not controlled during the development, may rise above the preferred ranges. Temperature requirements may be varied by one skilled in the art depending on the nature, characteristics, and the chemical components of the developing solution.

10. Yet, in another embodiment of the present invention, the solid support with latent pattern of probe-target complexes is exposed to a solution, containing a mixture of colloidal particles and an alternative binding agent. Said binding agent is repelled, i.e., not bound to the colloidal particles. Different reagents may be used as an alternative binding agent, including small organic molecules, biopolymers, including DNA, RNA, peptides, proteins, and the like. One particular example of such an alternative binding agent for use with gold colloids, which is given herein by way of illustration and not be way of limitation, is albumin molecules, and more specifically bovine serum albumin (BSA). In this embodiment of the invention, when the alternative binding agent binds to the surface of the solid support, it blocks the surface and prevent colloidal particles from binding to the same spot on the surface. The binding of the colloidal particles and alternative binding agent continue until the equilibrium is reached or until reagents are exhausted. In such an arrangement the density of colloidal material on the substrate surface represents the difference of the binding rate of the colloidal particles and the binding agent. Said difference of the binding rates usually is varied from site-to-site throughout the surface due to presence or absence of the probe-target complexes in the corresponded sites of the surface. Therefore, by measuring the density of colloidal material on the surface it is possible to identify location and measure the quantity of probe-target complexes on the surface.

11. The development step is carried out for a period adequate to develop the latent pattern satisfactorily. Usually about 2 to about 60 minutes, or preferably about 5 to about 30 minutes, will be sufficient. For optimal image development, one skilled in the art may vary the concentration of colloidal particles, the alternative binding agent, if such is present in solution, and temperature.

12. Yet, in another embodiment of the present invention the development solution is prepared by mixing a solution of colloidal particles and binding agent such as small organic molecules, biopolymers, peptides, proteins, and the like, in which the binding agent is capable binding to the substrate as well as to colloidal particles. One particular example of such binding agent for use with gold colloids, which is given herein by way of illustration and not be way of limitation, is poly-L-lysine molecules. In this embodiment of the invention, the rate of binding colloidal particles to the surface is given by the difference in the rate of binding of the agent to the colloidal particles and the rate of binding of the agent to the sites of the substrate. Said difference of the binding rates usually is varied from site-to-site throughout the surface due to presence or absence of the probe-target complexes in the corresponded sites of the surface. Therefore, by measuring the density of colloidal material precipitated on the surface it is possible to identify the location and measure the quantity of probe-target complexes on the surface. A new and unexpected result of the present embodiment of the invention is that the developing process is self-regulated. The development reaction is self-terminating and precipitation of the colloid stops when the binding agent saturates the colloidal particles and the substrate. This can be used to prevent the substrate from overdeveloping when exposing it to the developing solution for a substantially longer time than normal.

10. After development is complete, non-precipitated colloidal particles are removed by washing the substrate in an appropriate solvent or buffer solution, and most preferably in distilled deionized water.

13. The image of the colloidal material (i.e., colloidal particles) precipitated on substrate surface is captured using a conventional methods of capturing optical images such as a photocamera, an optical microscope equipped with a camera, or by using an optical scanner. For optimal image appearance, one skilled in the art may arrange different ways of illuminating substrate such that (a) the image is created due to light absorbing property of the colloidal particles; (b) the image is created by light specular reflected by substrate surface carrying precipitated colloidal particles; and (c) the image is created by light diffusely reflected by substrate surface carrying precipitated colloidal particles, see for example, Golovlev, et al, “Digital Imaging for Documenting and Modeling the Visual Appearance of 19th Century Daguerreotypes”, The J. Imaging Sci. and Technology, vol. 46, 1-7 (2002). It is contemplated that capturing the image created by specular reflectance will be the most beneficial when the size of individual colloidal particle is about 50 nm or smaller. It is considered to be more advantageous to capture image created by diffuse reflectance from the substrate surface when the size of the individual colloidal particle is about 50 nm or bigger. To capture mostly the diffuse reflectance from the substrate surface an opaque substrate can be employed, or equally acceptable, the back-side of the transparent substrate can be painted with a light absorbing paint or, equally acceptable, light absorbing screen can be placed behind the transparent substrate by employing an appropriately designed slide-carrying cassette. Said cassette comprising the light absorbing screen and means for maintaining the distance between the screen and the substrate surface. Here, the distance between the screen and the substrate surface must be bigger than a focal depth of the device employed to capture the image. The distance is usually not less than 1 mm and preferably more than 1 mm, and more preferably from 5 mm to 100 mm.

The method of current invention can be practiced using different types of substrates including glass, fused silica substrates, and substrates made of synthetic polymer materials, for instance polyethylene and its derivatives, polyethylene terephthalate (PET) and its derivatives, polyacrylamide and its derivatives, polymethacrylate and its derivatives, polysterene/divinylbenzene and its derivatives, and the like known in the art. One particular example of the synthetic polymer substrate, which is given here by way of illustration, and not be way of limitation, is Mylar™ polymer films. The Mylar polymer film has appealing surface properties. The polymeric surface is hydrophobic, which allows better control over the shape and size of printed microarray spots. At neutral pH the surface appears to be negatively charged and when exposed to a solution of colloidal gold it repels negatively charged gold particles. However, the surface can be modified and appear to be positively charged when treated with aγ-aminopropyltriethoxylsilane solution or exposed to a solution of poly-L-lysine. This modified Mylar film can capture gold particles from solution. When gold particles precipitate on the surface, the density of the particles can be quantitatively characterized by measuring the diffuse reflectance of the surface.

### EXAMPLE I

### Detection of Poly-L-Lysine on a Polymer Substrate

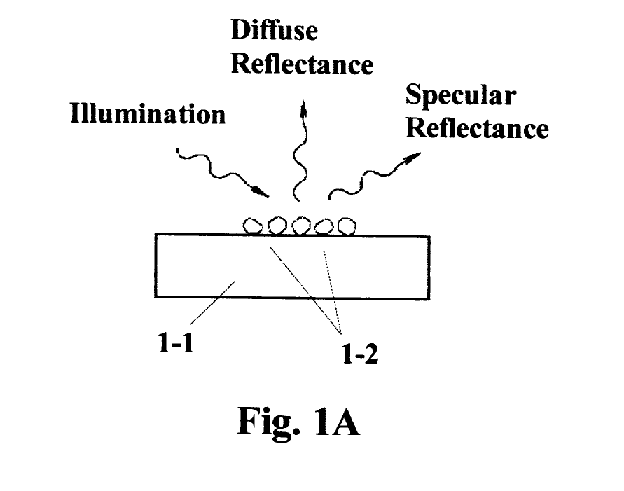

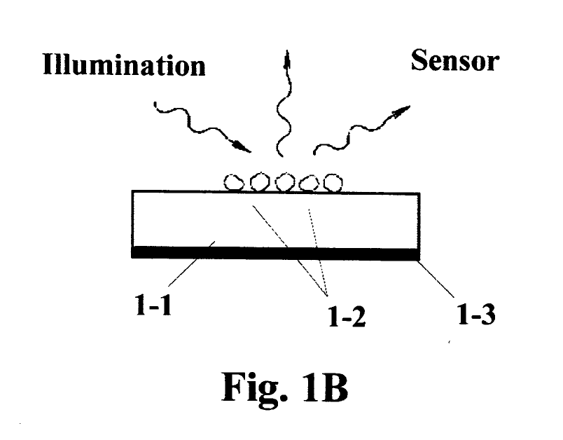

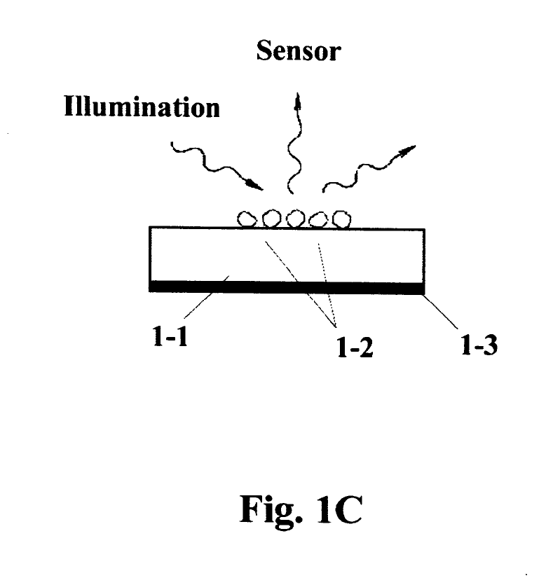

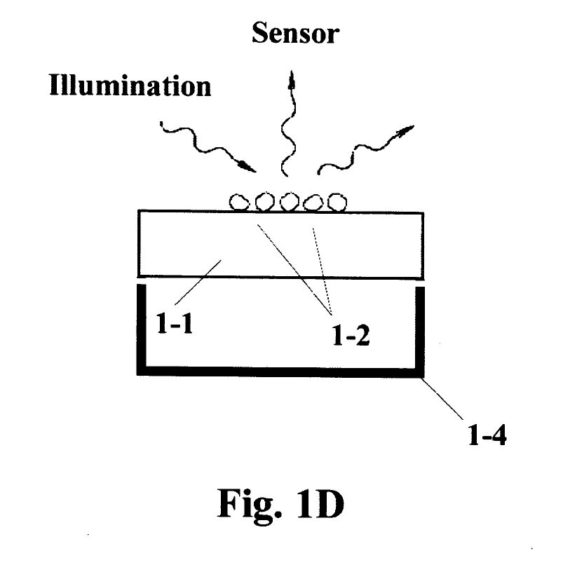

When gold particles on a surface are illuminated by external light source, the light is partially absorbed, specular reflected and diffusely reflected by colloidal particles precipitated on the substrate surface, as illustrated in FIG. 1A. In the visible spectral range the net reflected portion of the light normally dominates over the portion of the light absorbed by colloidal particles when the size of particles is 500 nm or less. Therefore, more sensitive detection of gold particles usually can be achieved by detecting reflected and scattered light vs. the measurement of the absorption. The exact ratio of the diffusely reflected component to the specular light component varies vs. the size of the particles. We observed that intensity of diffuse reflected light dominates over the reflected light when the size of gold particles is in the range of about 20 nm to 500 nm.

An optical flatbed scanner is particularly preferable for capturing light diffusely reflected by a surface. Two important components of the scanner are light source for illuminating the surface and a linear CCD element, which capture scattered light at some angle to the direction of illumination. In most commercially available scanners special measures are taken to minimize the specular reflected component captured by CCD element. To acquire image, the light source and CCD element move along the surface and capture pattern of the diffuse reflectance on the surface. This operation mode, e.g. front illumination mode, normally is used to capture images of paper documents. Most scanners are also equipped with a white lead screen and some scanners are equipped with additional diffuse light source for operating in a backside illumination mode, which is optimal for capturing prints produced on transparent substrates. In the back-illuminating mode light passes through the substrate, such as slide or film. Light captured by scanner sensor represents the sum of the absorbance and specular reflectance of the substrate. The backside illumination mode is particularly useful for detecting light-absorbing regions on the surface of transparent substrates and with the method of the present invention can be used for detection absorption of gold particles. However, we found that 50 nm, 100 nm and 250 nm gold particles provided a stronger signal and better signal-to-noise ratio when detected in front-illuminating mode by detecting diffusely reflected light.

To select a scanner for using with the method of present invention a number of parameters have to be taken into consideration, including: 1) Optical or true resolution, which for currently available commercial scanners is in the range from 1200 dpi to 6400 dpi; 2) The ability to export high dynamic range images, i.e, to export 16-bit gray or 48-bit color images; 3) The ability to operate either in front- or back-side illuminating mode; and 4) The rate of sending data to computer. We have selected Epson Perfection 3200 (Seiko Epson Co.) flatbed scanner which has an optical resolution up to 6400 dpi, can export 48-bit color images, can operates both in front- and back illuminating modes and provides data transfer rate up to 400 Mb/s. To take advantage of the capability of the scanner to make quantitative measurements, a TWAIN-compatible software was developed which allowed multiple scans and capture of any pre-defined number of scans of a specified region on the substrate. The multiple scans were used to accumulate signal and improve Signal-to-Noise (S/N) ratio for improving overall detection sensitivity. Different modes of capturing data were implemented for detection of 250 nm gold particles on opaque polymer substrate, blackened glass substrates, and transparent substrates in combination with light absorbing screen placed behind the substrate. In particular, the techniques include multiple scanning of the substrate surface and exploiting the benefit of capturing and processing high amount of information contained in high-resolution images.

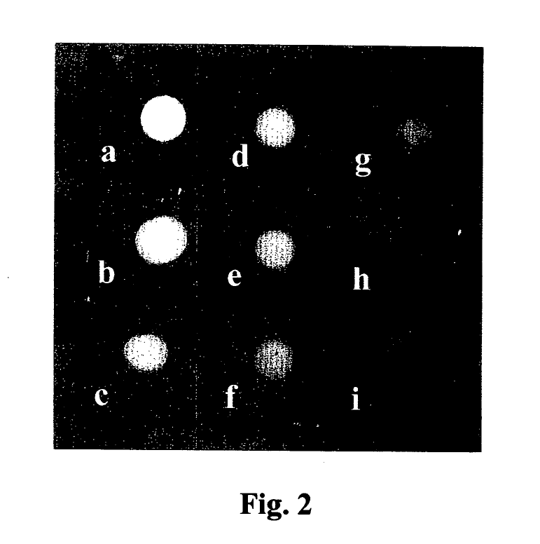

FIG. 2 show example of precipitating 250 nm gold particles (BBInternational, UK) on the surface of opaque Mylar film. In this illustration of the method of the present invention a latent pattern of spots of poly-L-lysine was first produced on the substrate by pipetting 1 μl of poly-L-lysine solution at concentrations (a) 1 ng/μl, (b) 0.8 ng/μl, (c) 0.6 ng/μl, (d) 0.5 ng/μl, (e) 0.4 ngμl, (f) 0.3 ng/μl, (g) 0.2 ng/μl, (h) 0.1 ng/μl, and (i) 0.05 ng/μl. The latent pattern was visualized by developing the substrate in solution of 250 nm gold particles at concentration 3.6×10⁸ ml⁻¹. The development was carried out at room temperature by dipping the substrate for 15 min into the solution of gold colloid. The size of the individual spot in FIG. 2 is 7.2 mm². The detection sensitivity achieved in this test is 7 pg/mm² of poly-L-lysine molecules per mm².

### EXAMPLE II

### Image Capturing Techniques for Increasing Signal-to-Noise (S/N) Ratio for Achieving Higher Detection Sensitivity

Enhancing S/N by averaging multiple scans: Averaging multiple scans increases S/N as the squareroot of the number of scans. This was observed and verified by capturing multiple scans of the image shown in FIG. 2. S/N was measured as the ratio of the average amplitude of the light diffusely reflected at the center of a spot labeled by gold to the average variation of the background signal in the close proximity to the spot. The same type of dependence of the S/N vs. number of scans was observed for 24-bit color images and 48-bit color images acquired using Epson Perfection 3200 scanner. For the same number of acquired scans S/N ration was higher for red and green component of the image and somewhat lower for the blue component of the color image of the gold particles. The last result is consistent with the yellowish color of the gold particle, since the red and green component of this color is higher than the corresponded blue component. For this reason, processing the red and green component of the image of gold particles and discarding the blue component of the image can usually provide a higher S/N ratio.

Enhancing S/N by reducing image size: Capturing an image at high-resolution and converting it to a lower resolution image often increases S/N. For instance, when the size, i.e., width and height, of the image is reduced twice, each block of 2×2, i.e., total of 4 pixels of the original image is “squeezed” into one pixel of the lower resolution image. When reducing the size of the image the amplitude of pixels of smaller image normally is calculated as average amplitude of a group of pixels from the original image. In the last example, reducing the size of the image twice can cause same effect on S/N as accumulating and averaging four lower resolution images, i.e., it increases S/N twice. Capturing image shown in FIG. 2 at resolutions of 800, 1200, 1600, 3200, and 6400 dpi and then reducing the size of the image to 600 dpi has confirmed this conclusion. In tests, the S/N was measured as the ratio of the amplitude of the light diffusely reflected at the center of a spot labeled by gold to the average variation of the background signal in the close proximity to the spot. As was discussed herein above, the S/N increases linear vs. the factor to which the size of the image was reduced and for image captured at 6400 dpi reducing the size to 600 dpi causes about 16-fold increase of S/N.

### EXAMPLE III

### Opaque and Blackened Substrates for Detection of Molecular Structures of Interest

Different types of substrates were employed with the method of present invention for immobilization of biopolymer molecules, including a glass and fused silica slides, polycarbonate polymers and Mylar™ polymer film. In tests, to minimize undesirable background signal from light passed through the substrate and reflected back by scanner lead or environment, a back surface of a transparent substrate was blackened by acrylamid-based paint (see FIG. 1B and FIG. 1C). Also were investigated substrates of polymer films known for production of magnetic floppy diskettes (Immation Co., MN). These opaque polymer films have smooth high quality surface covered by ˜1 μm film containing 10% of γ-Fe₂O₃ dispersed in a polymer matrix, see M. P. Sharrock, “Particulate Recording Media”, MRS Bulletin, March 1990, pp. 53-62. For improving mechanical property and chemical stability, the surface coating may include sub-micron particles of a telomer of tetrafluoroethylene, forced into the coating film by buffing and calendaring. The manufacturing process yields a very smooth high quality polymeric surface, which is mechanically and chemically stable. The surface can be further activated using known chemistries for modification polymers and was successfully used as a solid support for immobilization DNAs and protein molecules.

For immobilizing biopolymers on lysine coated surface the substrate was treated for 15 minutes in 0.1 % solution of poly-L-lysine (Sigma, MO), followed by washing in a distilled water. The substrate was dried in a flow of compressed filtered air. Amino-modified substrates for immobilizing biopolymers were prepared by exposing polymer or glass substrates to 1% solution of γ-aminopropyltriethoxylsilane in alcohol for 1 hour. Substrates were subsequently washed in distilled water, dried at room temperature and stored in desiccated container at room temperature.

### EXAMPLE IV

### Immobilization and Visualization of DNA and Proteins

Protein A (Cat. No. P6031), Human Immunoglobulin G from Serum (Cat. No. 14506), Bovine Serum Albumin (Cat. No. A7511), and single stranded 7,229 bases long M13mp8 Phage DNA (Cat. No. D8410) were purchased from Sigma, MO. A set of 11 monoclonal antibodies specific for DNA repair pathways was purchased from BD Biosciences (Cat. No. 611432). A 70 bases long synthetic oligonucleotides of different sequences with varied A-T vs. C-G composition were synthesized and PAGE-purified by AlphaDNA (Montreal, Quebec, Canada). For non-covalent immobilization (passive absorption) on substrate surface stock solution of each biopolymer was prepared at concentration of 100 ng/μl in distilled deionized water or, alternatively, in 0.3 M sodium acetate buffer (Sigma, MO, Cat. No. S-7899). For spotting corresponded biopolymers on substrate surface, freshly prepared stock solutions were diluted to a lower concentration of 10 ng/μl and 1 ng/μl as needed.

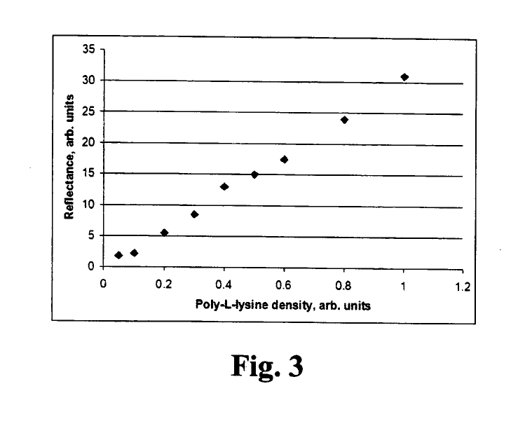

Detecting DNA. A single stranded 7,229 bases long Phage DNA (M13mp8) was immobilized on lysine coated Mylar substrate by pipetting 1 μL of each of three dilutions containing 100, 10, and 1 ng/μL of the DNA. To maintain absorption from solution and preventing spots from drying the substrate was incubated at room temperature overnight in humidified chamber. The substrate was thoroughly washed in distilled water to remove spotting solutions and unbound DNA molecules. The substrate with latent pattern of DNA spots was exposed for 30 minutes to solution of negatively charged 250 nm gold particles at concentration 3.6×10⁸ particles/ml. FIG. 3A shows image of the developed substrate captured by scanner. The substrate is uniformly covered by gold particles except for the spots where DNA was spotted at concentration 10 and 100 ng/μl. Thye absence of gold particles in these spots is consistent with the fact that DNA is carrying a net negative electric charge, which can repel negatively charged gold particles. To confirm that unstained spots in FIG. 3A indeed are caused by electrostatic repulsion of gold particles by negatively charged DNA, a solution of positively charged gold particles has been prepared by immobilizing poly-L-lysine on gold particles. Positively charged colloidal particles were prepared by adding 80 pμof 0.01% poly-L-lysine (Sigma-Aldrich) to 1 ml of gold colloid at concentration 3.6×10⁸ particles/ml. The mixture was incubated at room temperature at constant shaking for 2 hours. To remove unbound poly-L-lysine the solution was centrifuged to precipitate colloidal particles and the natant carrying the residual unbound poly-L-lysine was discarded. A pellet of colloidal particles was resuspended in distilled deionized water to the original concentration of 3.6×10⁸ particles/ml and this colloidal solution was used to develop the latent pattern of the DNA molecules on the substrate. The development was carried out by dipping the substrate into the colloidal solution for 15 min at room temperature. When development was completed, the substrate was washed gently using distilled deionized water, dried by centrifugation and scanned using Epson Perfection 3200 flatbed scanner.

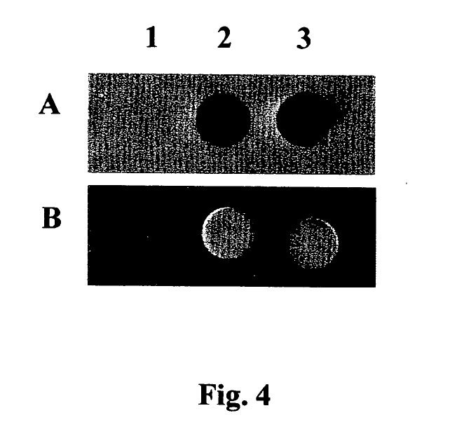

The positive charge on gold particles was confirmed first by applying to a lysine or amine coated substrate, on which no precipitation of gold particles on the substrate was observed. Next, the solution of positive gold particles was applied to the amino-modified polymer substrate carrying latent pattern of immobilized DNA as described herein above. The gold particles precipitated on sites where DNA was spotted. A corresponded image of the substrate is shown in FIG. 4B where the image resembles a “negative image” of the spots in FIG. 4A. This is consistent with the proposed mechanism of electrostatic interaction of charged gold particles and molecular structures on the substrate surface.

The total quantity of DNA immobilized on surface in spot #2 does not exceeded 10 ng, and by taking into account that the area of individual spots in FIG. 3 is 7.2 mm², one can estimate the sensitivity of the present method of DNA detection as less than (10 ng/7 mm²)=1.4 ng/mm². When projected to a spot having the size of 160 μm in diameter, which can be easily detected by scanner, this detection sensitivity corresponds to 28 μg of DNA per spot.

Reducing gold binding capacity of activated surfaces. Competitive labeling of biopolymers on a surface. In some cases it might be desirable to block or cap activated surface and prevent binding gold particles which otherwise occur all over the substrate surface. Different blocking reagents and chemistries were reported in the literature, including the use of acetic anhydride for blocking amine groups on an amine terminal spacer and glycine for blocking poly-L-lysine coated surfaces. In addition to the methods known in the art, in the method of the present invention exposing activated surfaces to a solution of serum albumin (BSA) modifies and reduces binding capacity of the surface. By choosing an appropriate exposure time and by adjusting the concentration of the albumin molecules in solution it is possible to achieve a partial or complete blocking of the surface against precipitation of gold particles. The approach of blocking the surface by albumin has been used by the method of the present invention for competitive labeling of molecular structures on a substrate surface. To illustrate this approach spots of poly-L-lysine were printed on amino-modified Mylar surface as presented herein above in Example I. When the substrate was developed in a colloidal solution of 250 nm negatively charged gold particles at a concentration of 3.6×10⁸ particles/ml for 30 minutes, the substrate surface was saturated and uniformly covered by gold particles. Indeed, both polylysine molecules and amino groups are bound to gold particles and no polylysine spots on the surface could be identified. However, when an amino-modified substrate with spots of polylysine was developed in a colloidal solution containing 3% of Bovine Serum Albumine (Sigma, MO), the average density of bound gold particles was significantly reduced everywhere except for the spots on the surface covered by poly-L-lysine. The spots can be easily identified and quantitatively characterized using the image captured by a scanner. Indeed, when both colloidal particles and albumin molecules are added to the developing solution, the molecules and particles are competing for the bind sites on the substrate surface. Absorption rate of albumin on amino-modified surface may be higher then in the region covered by poly-L-lysine. This causes the surface to be blocked faster by albumin everywhere on the surface except for the spots covered by poly-L-lysine. The difference in the reaction rate is projected into the variation of the density of precipitated gold particles on the surface and can be used to discriminate spots of different biopolymers based on the difference in the rate of attaching colloidal particles and an alternative binding agent such as BSA.

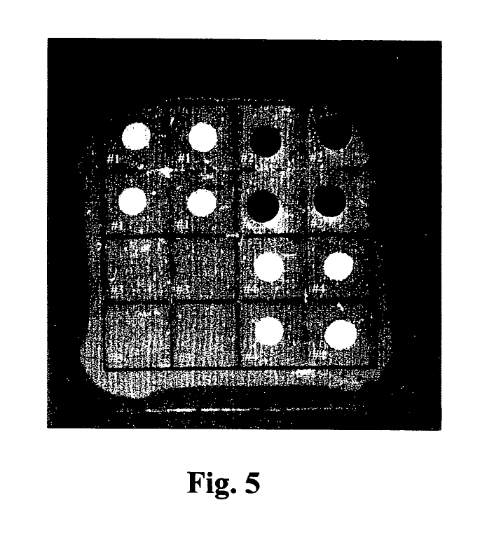

Detecting protein A and Immunoglobulin G. Different biopolymers may have different affinities to colloidal gold. When immobilized on a surface, such biopolymers increase or reduce gold binding capacity of the surface and can be detected by measuring the amount of gold particles precipitated on the surface. Two examples, which can illustrate this approach, are detection of protein A and Immunoglobulin G (ImG). When immobilized through passive adsorption on amino-modified Mylar substrate, the first, protein A, decreases and the second, ImG, increase gold binding capacity of the substrate. The latent pattern of proteins on the substrate was prepared by spotting 1 μl of 100 ng/μl solution of Protein A and Immunoglobulin G in 0.3M sodium acetate buffer and by incubating the substrate overnight in humidified chamber. Next, part of the substrate carrying twospots of the Protein A (see spot #4 in FIG. 5) was incubated for 1 hour at room temperature in a solution of ImG at a concentration 100 ng/μl. The substrate was washed in distilled deionized water, dried and developed in solution of 250 nm negatively charged gold particles at concentration 3.6×10⁸ particles/ml for 5 minutes. The relatively short developing time was used to avoid saturation and reduce the density of precipitated gold particles below the maximum gold binding capacity of the substrate. Under such conditions, the density of gold particles in the spot covered by ImG is higher than the background density (see spots #1 in FIG. 5) and the density in the spot covered by Protein A is lower than the background density of gold particles (see spots #2 in FIG. 5). The Protein A and ImG are capable to bind and form probe-target complex upon interaction. This can be observed in spots #4 in FIG. 5, where reducing of the gold binding capacity of the substrate due to immobilization of Protein A is overcome by increasing the binding capacity due to attachment of ImG at sites where Protein A is immobilized.

A set of dilutions was used to immobilize different quantity of ImG on substrate and determine the detection sensitivity for ImG. Consistently with what was previously observed for DNA and poly-L-lysine, ImG spots with density of 2.1 ng/mm² were detectable and show S/N >3 in an image captured after a single scan of the substrate surface. An increase of binding capacity of the surface and similar detectable density of antibodies of about 2.0 ng/mm² were observed for a set of 10 monoclonal antibodies specific for DNA repair pathways (BD Biosciences, CA, Cat. No. 611432).

### EXAMPLE V

### Detection of Hybridized Nucleic Acids

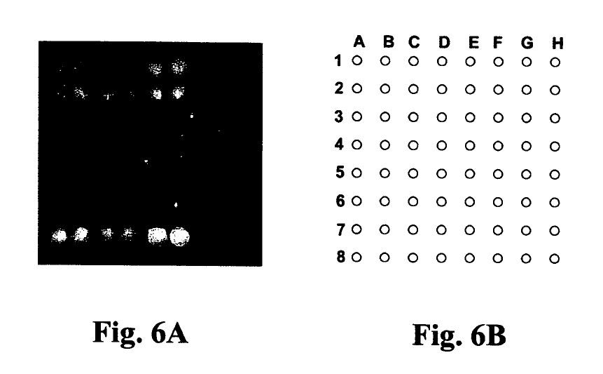

A set of 5 oligonucleotides each 50 bases long was printed on surface of amino-modified glass substrate (Corning Co.) by following the manufacturer protocol (Corning Co., User guide). Oligonucleotides were printed in an array of 8×8 spots with layout shown in FIG. 6B. Oligonucleotide sequence, concentration of printing solution and location of corresponded spots are shown in Tables I and II. The microarray was printed using SpotBot array Printer (TeleChem International, CA).

TABLE I
Sequence of probes used to produce
microarray shown in FIG. 6.

| No. | Probe | Sequence |
|---|---|---|
| 1 | RPL6 | 5′-tcgc aaaa tgcc taga tatt atcc tact gaag atgt gcct cgaa agct gttg agcc |
| | | acgg-3′ |
| 2 | TAF2H | 5′-ggcc aacg gaga cgtg aagc ccgt ggtg tcca gcac gcct ttgg tgga cttc ttga |
| | | tgca-3′ |
| 3 | COX4 | 5′-ccag gtca cctt gggc tctg tttg tcag atcc tgtt atcc atag cctt taga gagg |
| | | acct-3′ |
| 4 | HSPCB | 5′-agaa gatc agac agag tacc taga agag aggc gggt caaa gaag tagt gaag |
| | | aagc attc-3′ |
| 5 | L28 | 5′-cacc aaga gctc ctga gccc cctg cccc caga gcaa taaa gtca gctg gctt tctc |
| | | aaaa-3′ |

Printed microarray was incubated in 0.01% solution of poly-L-lysine for 30 min at room temperature and then blocked first for 20 min in 30 ml PBS buffer containing 75 mg of sodium borohydrate and 6.5 ml of ethanol, and then for 20 min in 4% BSA (bovine serum albumin) aqueous solution at 42° C. The microarray was hybridized with a sample substance containing a total 200 ng of oligonucleotides homologous to the probes 1-4 (see Table I). The hybridization mix was prepared in ×1 SSC buffer (Sigma-Aldrich, MO) and incubated with the microarray overnight at 42° C. After a stringency wash, the microarray was developed for 30 min in solution of 250 nm positive gold particles prepared according to the procedure described hereinabove: 80 μl of 0.01% poly-L-lysine (Sigma-Aldrich) was added to 1 ml of 250 nm gold colloid at concentration 3.6×10⁸ particles/ml. The mixture was incubated at room temperature at constant shaking for 2 hours. The solution was centrifuged to precipitate colloidal particles and the natant carrying the residual unbound poly-L-lysine was discarded. A pellet of colloidal particles was resuspended in distilled deionized water to the original concentration of 3.6×10⁸ particles/ml and this colloidal solution was used to develop the latent pattern of the hybridized oligonucleotides. When development was completed, the substrate was washed gently using distilled deionized water, dried by centrifugation and scanned using Epson Perfection 3200 flatbed scanner. The microarray image is shown in FIG. 6A. All four homologous target oligos can be identified on this image, although consistently, no

TABLE II
Layout of microarray and concentration of spotting
buffers used to produce microarray in FIG. 6

| | A | B | C | D | E | F | G | H |
|---|---|---|---|---|---|---|---|---|
| 1 | RPL6 | RPL6 | TAF2H | TAF2H | COX4 | COX4 | HSPCB | HSPCB |
| | 20 μM | 20 μM | 20 μM | 20 μM | 20 μM | 20 μM | 20 μM | 20 μM |
| 2 | RPL6 | RPL6 | TAF2H | TAF2H | COX4 | COX4 | HSPCB | HSPCB |
| | 10 μM | 10 μM | 10 μM | 10 μM | 10 μM | 10 μM | 10 μM | 10 μM |
| 3 | L28 | L28 | L28 | L28 | Buffer | Buffer | Buffer | Buffer |
| | 1.25 μM | 1.25 μM | 1.25 μM | 1.25 μM | | | | |
| 4 | RPL6 | RPL6 | TAF2H | TAF2H | COX4 | COX4 | HSPCB | HSPCB |
| | 1.25 μM | 1.25 μM | 1.25 μM | 1.25 μM | 1.25 μM | 1.25 μM | 1.25 μM | 1.25 μM |
| 5 | RPL6 | RPL6 | TAF2H | TAF2H | COX4 | COX4 | HSPCB | HSPCB |
| | 0.5 μM | 0.5 μM | 0.5 μM | 0.5 μM | 0.5 μM | 0.5 μM | 0.5 μM | 0.5 μM |
| 6 | RPL6 | RPL6 | TAF2H | TAF2H | COX4 | COX4 | HSPCB | HSPCB |
| | 0.1 μM | 0.1 μM | 0.1 μM | 0.1 μM | 0.1 μM | 0.1 μM | 0.1 μM | 0.1 μM |
| 7 | RPL6 | RPL6 | TAF2H | TAF2H | COX4 | COX4 | HSPCB | HSPCB |
| | 0.05 μM | 0.05 μM | 0.05 μM | 0.05 μM | 0.05 μM | 0.05 μM | 0.05 μM | 0.05 μM |
| 8 | RPL6 | RPL6 | TAF2H | TAF2H | COX4 | COX4 | HSPCB | HSPCB |
| | 5 μM | 5 μM | 5 μM | 5 μM | 5 μM | 5 μM | 5 μM | 5 μM |

hybridization can be detected in 8 spots representing a negative control spots (see spots A1-A8 in FIG. 7A). This example therefore illustrates a method of the present invention for practice with hybridization microarrays known in the art.

### EXAMPLE VI

### Enzymatic Digestion for Improving Discrimination fSites Carrying Hybridized or Non-Hybridized Molecular Structures

Some applications, such as detection of Single Nucleotide Polymorphisms or identification of extremely low quantity of target species in a sample substance may require advanced discrimination level of sites where probe and target were hybridized vs. the sites with no hybridization. Enhancing discrimination between such sites can be achieved by employing enzymatic digestion of probes, or alternatively probe-target complexes, such that only hybridized, or alternatively only non-hybridized, molecular structures will remain on the substrate and will be detected by labeling with colloidal particles as disclosed herein above. In this embodiment of the present invention microarray first hybridized with target molecular structures of a sample substance and after a stringency wash is exposed to a solution containing S1 nuclease isolated from Aspergillus oryzae (Stratagene). The solution can contain from a fraction of 1 to 200 units of the S1 nuclease in a buffer composed of 20-300 mM sodium acetate, 0-5% glycerole (v/v), 0.1-2.8 M NaCl and 0.1-10 mM ZnSO₄. To achieve the desirable effect of degrading unbound probe molecules on the substrate the microarray is incubated in this digestion mix from 1 min to 24 hours at temperature ranging from 15° C. to 45° C. and the solution pH adjusted to the range of about 3 to 10. One skilled in the art will adjust composition of the digestion mix and treatment conditions to achieve satisfactory result. In this example, the S1 nuclease degrades single-stranded nucleic acids on the substrate and efficiently eliminates unbound probes, which otherwise may be a source of false positive identification of hybridized probe-target complexes on the microarray.

We claim:

1. A method of detecting a latent pattern of molecular structures on solid support comprising:
providing a type of colloidal particles, each particle carrying a net electric charge and is capable of electrostatic interaction with the solid support and molecular structures of interest;
contacting the colloidal particles and the solid support on which said latent pattern of molecular structures need be detected; and
observing a detectable change brought about by colloidal particles precipitated on the surface of the solid support such that the density of precipitated colloidal particles on the surface follows the pattern of the molecular structures which need be detected.

2. The method of claim 1 wherein the latent pattern of molecular structures is formed by hybridized nucleic acids.

3. The method of claim 1 wherein the latent pattern of molecular structures is resulted from specific binding of target and probe proteins.

4. The method of claim 1 wherein colloidal particles having size of less then 10 μM, and more preferably having the size of less then 1 μm.

5. The method of claim 1 wherein colloidal particles are essentially gold nanoparticles.

6. The method of claim 1 wherein colloidal particle carrying a net negative electric charge.

7. The method of claim 1 wherein colloidal particles are covered by a positively charged polymer substance and therefore are carrying a net positive electric charge in solutions with widely varied pH value.

8. The method of claim 1 wherein surface of solid support additionally is treated in a solution of a positively charged polymer substance.

9. The method of claim 1 wherein surface of solid support additionally is treated in a solution of a negatively charged polymer substance.

10. The method of claim 1 wherein latent pattern of molecular structures is created by enzymatic digestion of molecular structures on the surface of the solid support.

11. A method of detecting a latent pattern of molecular structures on the solid support comprising:
providing a type of colloidal particles, each particle carrying a net electric charge and is capable for electrostatic interaction with the solid support and molecular structures of interest;
providing an alternative binding agent, which agent can be absorbed on the surface of the solid support and, when absorbed on the surface, said agent prevent colloidal particles from precipitating in corresponding sites of the solid support;
contacting the mixture of colloidal particles and said alternative binding agent with the solid support on which said latent pattern of molecular structures need be detected; and
observing a detectable change brought about by colloidal particles precipitated on the surface of the solid support such that density of precipitated colloidal particles on the surface follows the pattern of the molecular structures and is given by difference of binding rate of colloidal particles and the absorption rate of the alternative binding agent to corresponding sites of the solid support.

12. A method of detecting a latent pattern of molecular structures on the solid support comprising:
providing a type of colloidal particles, each particle carrying a net electric charge and is capable for electrostatic interaction with the solid support and molecular structures of interest;
providing an alternative binding agent, which agent can bind either to the surface of the solid support or to the colloidal particles, and which agent when bind to the surface prevents colloidal particles carrying the same binding agent from precipitating in corresponding sites of the support;
contacting the mixture of colloidal particles and said alternative binding agent with the solid support on which said latent pattern of molecular structures need be detected; and
observing a detectable change brought about by colloidal particles precipitated on the surface of the solid support such that density of precipitated colloidal particles on the surface follows the pattern of the molecular structures and is given by difference of the absorption rate of the agent to colloidal particles and corresponding sites of the solid support.

13. A method of detecting a latent pattern of molecular structures on the solid support comprising:
providing opaque solid support or equally acceptable, providing a transparent solid support with back-side blackened by light absorbing paint;
creating the latent pattern of molecular structures on the solid support by immobilizing probe molecular structures in multiple sites on the solid support and binding the probe molecular structures with target molecular structures in a sample substance;
visualizing the latent pattern by precipitating colloidal particles on the surface of solid support and capturing image of diffusely reflected light from the surface of the support.

14. A method of detecting a latent pattern of molecular structures on the solid support comprising:
providing transparent solid support and a light absorbing screen, and means, such as a cassette, of keeping the screen on predetermined distance behind the transparent support;
creating the latent pattern of molecular structures on the solid support by immobilizing probe molecular structures in multiple test sites on the solid support and binding the probe molecular structures with target molecular structures by exposing the solid support to a sample substance;
visualizing the latent pattern by precipitating colloidal particles on the surface of solid support and capturing image of diffusely reflected light from the surface of the support.
